(19) United States
(12) Patent Application Publication
(10) Pub. No.: US 2026/0108692 A1
(43) Pub. Date: Apr. 23, 2026

(54) INTUBATION TUBE HOLDER

(71) Applicants: NANJING DRUM TOWER HOSPITAL, Nanjing, Jiangsu (CN); NANJING TECH UNIVERSITY, Nanjing, Jiangsu (CN)

(72) Inventors: Can XU, Nanjing, Jiangsu (CN); Luyang ZHOU, Nanjing, Jiangsu (CN); Xuewen ZHU, Nanjing, Jiangsu (CN); Kuikui ZHANG, Nanjing, Jiangsu (CN)

(21) Appl. No.: 19/121,654

(22) PCT Filed: Aug. 26, 2024
(86) PCT No.: PCT/CN2024/114405

§ 371 Date: Apr. 16, 2025

(30) Foreign Application Priority Data
Apr. 26, 2024 (CN) 202410511043.X

(51) Int. Cl. A61M 16/04

(52) U.S. Cl. CPC A61M 16/0497; A61M 16/0493

(57) ABSTRACT

The present disclosure belongs to the technical field of medical instruments, and discloses an intubation tube holder, including a clamping sleeve. A mounting notch is provided in each of two sides of one end of the clamping sleeve. The two mounting notches are symmetrically distributed about a circle center of the clamping sleeve. A support plate is rotatably connected into each of the two mounting notches. Elastic dental pads are fixedly connected to upper and lower ends of the support plates. The support plates are driven to rotate to original positions through elastic release of torsional springs. The two support plates move to a periphery to drive the elastic dental pads to move, so that the elastic dental pads on the two sides are just located between upper and lower groups of chewing teeth of a patient. When the mandible muscles of the patient naturally contract, the upper and lower chewing teeth of the patient are just in contact with the elastic dental pads at tops and bottoms of the support plates. The elastic dental pads play a role of supporting the mouth of the patient. Compared with an existing holder, the intubation tube holder solves the following problem that the patient bites on an outer wall of the holder with the front teeth for a long time, and further avoids looseness and deformation due to overtime stress on the front teeth of the patient.

### TECHNICAL FIELD

The present disclosure relates to the technical field of medical instruments, and in particular, to an intubation tube holder.

### BACKGROUND

Intubation is to insert a catheter into a human body through a body surface and a lumen inside the body, to input and extract gas or liquid. Common intubation includes endotracheal intubation (inserting a tube into a human trachea through a pharynx and a glottis for ventilation), and gastric intubation (inserting a tube into a human stomach through an esophagus to extract gastric juice or input a nutrient solution). Typically, after intubation, a holder needs to be used to secure the tube to ensure its stability.

Some existing intubation tube holders lack an ideal support structure for the mouth of a patient during use. Due to the natural contraction of the mandible muscles of the patient, the patient bites on an outer wall of the holder with the front teeth for a long time. Due to overtime stress on the front teeth, the front teeth easily get loose, causing discomfort in the patient.

### SUMMARY

In view of the shortcomings of the existing technology, the present disclosure aims to provide an intubation tube holder. Support plates are driven to rotate to original positions through elastic release of torsional springs. Two support plates move to a periphery to drive elastic dental pads to move, so that the elastic dental pads on two sides are just located between upper and lower groups of chewing teeth of a patient. When the mandible muscles of the patient naturally contract, the upper and lower chewing teeth of the patient are just in contact with the elastic dental pads at tops and bottoms of the support plates. The elastic dental pads play a role of supporting the mouth of the patient. Compared with an existing holder, this intubation tube holder solves the following problem that the patient bites on an outer wall of the holder with the front teeth for a long time, so that looseness and deformation due to overtime stress on the front teeth of the patient.

The objective of the present disclosure can be achieved by the following technical solution:

An intubation tube holder includes:
a clamping sleeve, wherein a mounting notch is provided in each of two sides of one end of the clamping sleeve; the two mounting notches are symmetrically distributed about a circle center of the clamping sleeve; a support plate is rotatably connected into each of the two mounting notches elastic dental pads are fixedly connected to upper and lower ends of the support plates;
hinge bars are vertically rotatably connected into the mounting notches; one end of each support plate is fixedly connected to a side wall of each hinge bar; torsional springs sleeve the hinge bars at the upper and lower ends of the support plates; one end of each of the two torsional springs close to each support plate is fixedly connected to the support plate, and the other end is respectively fixedly connected to a top wall and bottom wall of each mounting notch;
curved gears are fixedly connected to side walls of the two hinge bars; racks are meshed with peripheries of the two curved gears; the two racks are both located between the curved gears and inner walls of the mounting notches; first sliding chutes communicated with the mounting notches are provided inside side walls of two sides of the clamping sleeve in a length direction of the clamping sleeve; linkage bars are slidably connected in the two first sliding chutes; and end portions of the two linkage bars are fixedly connected to end portions of the racks corresponding to the two linkage bars.

In a preferred solution of the present disclosure, one end, away from each rack, of each of the two linkage bars is fixedly connected with a lever, and the levers are perpendicular to the linkage bars; second sliding chutes communicated to insides of the first sliding chutes are provided in two sides of a side wall of the clamping sleeve; the two levers are slidably connected into the corresponding second sliding chutes; outer side ends of the two levers extend to outer sides of the second sliding chutes; the outer side ends of the two levers are jointly connected with one arc-shaped connection frame; and the arc-shaped connection frame is located at a top periphery of the clamping sleeve.

In a preferred solution of the present disclosure, the two support plates are symmetrically distributed about the circle center of the clamping sleeve; and the two support plates are arranged in an arc manner.

In a preferred solution of the present disclosure, the elastic dental pads are made of a soft silicon material.

In a preferred solution of the present disclosure, two symmetrically distributed clamping blocks are provided inside the clamping sleeve; two clamping ejector rods distributed above and below are fixedly connected to side surfaces of the two clamping blocks far away from each other; the two clamping ejector rods slide through the clamping sleeve, and extend into the mounting notches; one end, away from each clamping block, of each of the two clamping ejector rods is fixedly connected with a thrust base; one side surface of each thrust base close to the clamping block is fixedly connected with a return spring; each return spring sleeves a periphery of each clamping ejector rod; one end of each return spring away from each thrust base is fixedly connected to a side wall of each mounting notch;
upper and lower side surfaces, close to each other, of the two thrust bases on the same side are rotatably connected with guide wheels; upper and lower ends of the curved gears are fixedly connected with cams; and the two cams are respectively in one-to-one correspondence to positions of the two guide wheels.

In a preferred solution of the present disclosure, the two clamping blocks are symmetrically distributed about the circle center of the clamping sleeve, and the two clamping blocks are arranged in an arc manner.

In a preferred solution of the present disclosure, a water supplement pressure plate is slidably connected to the periphery of the clamping sleeve; one side surface of the water supplement pressure plate close to the support plates is fixedly connected with a rubber bag for water supplement; the rubber bag for water supplement is located at an edge of the water supplement pressure plate; several small holes for water supplement are provided in one side surface of the rubber bag for water supplement close to the support plates; the several small holes for water supplement are communicated to an inside of the rubber bag for water supplement; the rubber bag for water supplement is filled with a water absorption sponge block;
a circular slot is provided in an upper part of the water supplement pressure plate in a penetrating manner; a water injection tube is fixedly connected in the circular slot; a water supplement hole coaxial with the circular slot is provided in an upper part of one side surface of the rubber bag for water supplement close to the water supplement pressure plate; an inner diameter of the water supplement hole is less than an inner diameter of the circular slot; an inner side end of the water injection tube is fixedly connected to the rubber bag for water supplement around the water supplement hole; and a rubber plug is embedded into the water injection tube.

In a preferred solution of the present disclosure, the water supplement pressure plate is arranged in an arc manner.

In a preferred solution of the present disclosure, a plurality of rebound seats distributed at equal angles are fixedly connected to an outer wall of the clamping sleeve; the rebound seats are located on one side of the water supplement pressure plate away from the rubber bag for water supplement; one side surface of each of the plurality of rebound seats close to the water supplement pressure plate is fixedly connected with a rebound rubber band; one end, away from each rebound seat, of each of the plurality of rebound rubber bands is fixedly connected to a surface of the water supplement pressure plate.

In a preferred solution of the present disclosure, the rubber bag for water supplement is made of a highly elastic rubber material.

Nouns, conjunctions, or adjectives involved in the foregoing technical solutions are explained as follows:

Fixed connection means connection without any relative movement after parts or components are fixed. The fixed connection includes detachable connection and non-detachable connection.
(1) The detachable connection is to fix parts together by using a screw rod, a spline, a wedge pin, and the like. In this connection mode, parts can be removed during maintenance, without damage. However, connection pieces used have be correct in specification (such as lengths of a bolt, a key, and a wedge pin), and are properly fastened.
(2) The non-detachable connection mainly means soldering, riveting, over-tenon fitting, and the like. Since a part cannot be removed until it is forged, sawed, or oxygen-cut during repair or replacement, usually, the part cannot be used again. In addition, during connection, attention should be paid to process quality, technical detection, and remedial measures (such as correction and polishing).

Threaded connection means detachable connection for connecting a connected member as a whole by using a threaded member (or a threaded portion of the connected member).

Sliding connection means that two objects are in contact with each other, but are not fixed, and the they can slide relative to reach other.

Rotatable connection means connection between parts to cause the parts to rotate with each other.

The present disclosure has the following beneficial effects:
1. In the present disclosure, the arc-shaped connection frame is pulled to drive the two levers to slide along the second sliding chutes. The two levers slide to drive the two linkage bars to slide along the first sliding chutes, thus further driving the two racks to move. When moving, the two racks drive the curved gears meshed with the two racks to rotate, so as to drive the two support plates to rotate along a hinge position between the hinge bars. In this case, the torsional springs undergo torsional deformation for force storage, so that the two symmetrically distributed support plates and elastic dental pads are contracted to the middle, and it is convenient to place the holder in the oral cavity of a patient, thereby improving the convenience of mounting of the holder.
2. In the present disclosure, by withdrawing a pulling force on the arc-shaped connection frame, the torsional springs are elastically released to drive the support plates to rotate towards the outside to original positions along connection positions for the hinge bars and the mounting notches. The two support plates move to a periphery to drive the elastic dental pads to move, so that the elastic dental pads on the two sides are just located between upper and lower groups of chewing teeth of the patient. When a medical staff withdraws an opening force on the mouth of the patient, the mandible muscles of the patient naturally contract, causing the upper and lower chewing teeth of the patient to be just in contact with the elastic dental pads at tops and bottoms of the support plates. The elastic dental pads play a role of supporting the mouth of the patient. Compared with an existing holder, the intubation tube holder solves the following problem that the patient bites on an outer wall of the holder with the front teeth for a long time, and further avoids looseness and deformation due to overtime stress on the front teeth of the patient.
3. In the present disclosure, when the support plates are driven to rotate towards two sides through the torsional springs, the cams connected to the curved gears are driven to rotate together, so that the cams push the guide wheels to move towards the middle of the clamping sleeve. The movement of the guide wheels drives the thrust bases and the clamping ejector rods to move towards the middle along slidable connection positions of the clamping sleeve, which further pushes the clamping blocks to move, so that the two symmetrically distributed clamping blocks move towards the middle and then automatically complete clamping on an intubation tube. Compared with the existing holder, the present disclosure greatly improves ease of use of this holder.
4. In the present disclosure, the water supplement pressure plate slides towards the lips of the patient. At the same time, the plurality of rebound rubber bands are elastically pulled. The movement of the water supplement pressure plate drives the rubber bag for water supplement and the water absorption sponge block to move together, until the rubber bag for water supplement is in contact with the lips of the patient. The rubber bag for water supplement is compressed to elastically collapse to press the water absorption sponge block inside the rubber bag for water supplement, and pure water contained inside the water absorption sponge block is extruded out through the plurality of small holes for water supplement, to moisturize the lips of the patient. Compared with water supplement by using a cotton swab, this greatly improves convenience and efficiency. After the moisturizing on the lips of the patient is completed, the water supplement pressure plate is loosened, and a rebound force of the plurality of rebound rubber bands drives the water supplement pressure plate and the rubber bag for water supplement to slide and return along the outer wall of the clamping sleeve, thereby automatically releasing pressure of the rubber bag for water supplement on the lips of the patient.

### BRIEF DESCRIPTION OF THE DRAWINGS

To describe the technical solutions in the embodiments of the present invention or in the related art more clearly, the following briefly introduces the accompanying drawings for describing the embodiments or the related art. A person of ordinary skill in the art may still derive other drawings from the accompanying drawings without creative efforts.

FIG. 1 is a schematic structural diagram I of the present disclosure;

FIG. 2 is a schematic structural diagram II of the present disclosure;

FIG. 3 is a schematic structural diagram III of the present disclosure;

FIG. 4 is an enlarged schematic structural diagram of part A of FIG. 3 according to the present disclosure;

FIG. 5 is a schematic diagram of a cross-sectional structure of a lip moisturizing component according to the present disclosure;

FIG. 6 is an enlarged schematic structural diagram of part B of FIG. 5 according to the present disclosure;

FIG. 7 is a schematic diagram of a cross-sectional structure of a clamping sleeve according to the present disclosure; and

FIG. 8 is an enlarged schematic structural diagram of part C of FIG. 7 according to the present disclosure.

In the drawings: 1: clamping sleeve; 101: mounting notch; 102: first sliding chute; 103: second sliding chute; 201: hinge bar; 202: support plate; 203: elastic dental pad; 204: torsional spring; 205: curved gear; 206: rack; 207: linkage bar; 208: lever; 209: arc-shaped connection frame; 301: clamping block; 302: clamping ejector rod; 303: return spring; 304: thrust base; 305: guide wheel; 306: cam; 401: water supplement pressure plate; 402: rubber bag for water supplement; 403: small hole for water supplement; 404: water absorption sponge block; 405: circular slot; 406: water injection tube; 407: rubber plug; 501: rebound seat; and 502: rebound rubber band.

### DETAILED DESCRIPTION

The following clearly and completely describes the technical solutions in the embodiments of the present disclosure with reference to the accompanying drawings in the embodiments of the present disclosure. Apparently, the described embodiments are some of the embodiments of the present disclosure rather than all of the embodiments. All other embodiments obtained by a person of ordinary skill in the art based on the embodiments of the present disclosure without creative efforts shall fall within the protection scope of the present disclosure.

In the description of the present disclosure, it should be understood that orientation or position relationships indicated by the terms such as “hole”, “above”, “below”, “thickness”, “top”, “middle”, “length”, “inside”, and “around” are used only for ease and brevity of illustration and description, rather than indicating or implying that the mentioned component or element need to have a particular orientation or need to be constructed and operated in a particular orientation. Therefore, such terms should not be construed as limiting of the present disclosure.

Some existing intubation tube holders lack an ideal support structure for the mouth of a patient during use. Due to the natural contraction of the mandible muscles of the patient, the patient bites on an outer wall of the holder with the front teeth for a long time. Due to overtime stress on the front teeth, the front teeth easily get loose, causing discomfort in the patient. In addition, some existing holders do not have a function of moisturizing the lips of a patient, and a nurse needs to dip a cotton swab in water and moisturize the lips of the patient with the dipped cotton swab. The operation is more inconvenient.

To solve the foregoing two problems, the following solutions may be used.

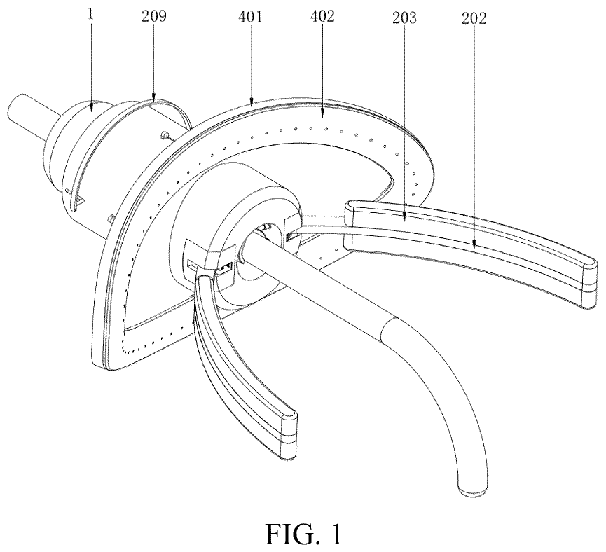

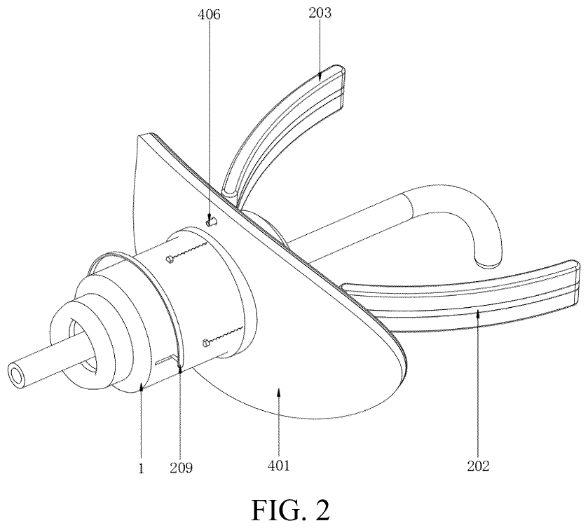

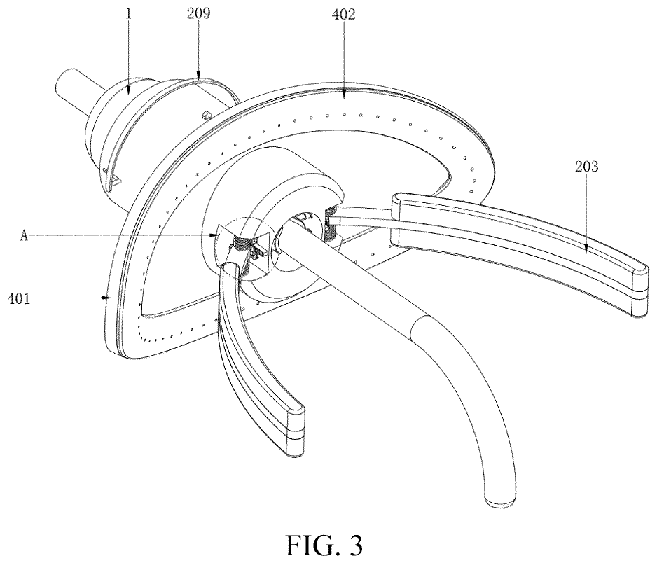

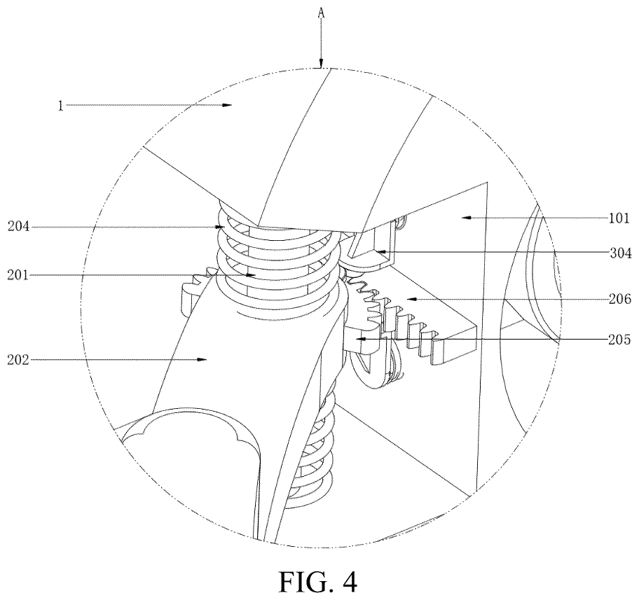

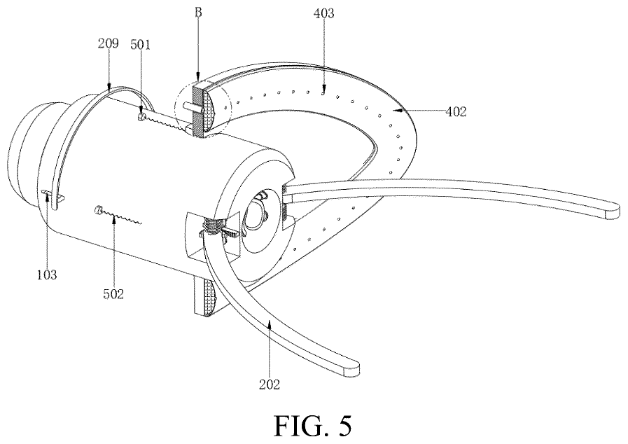

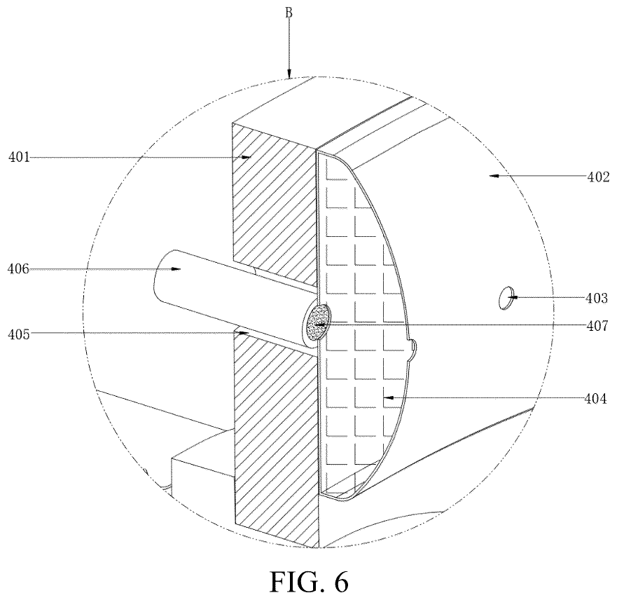

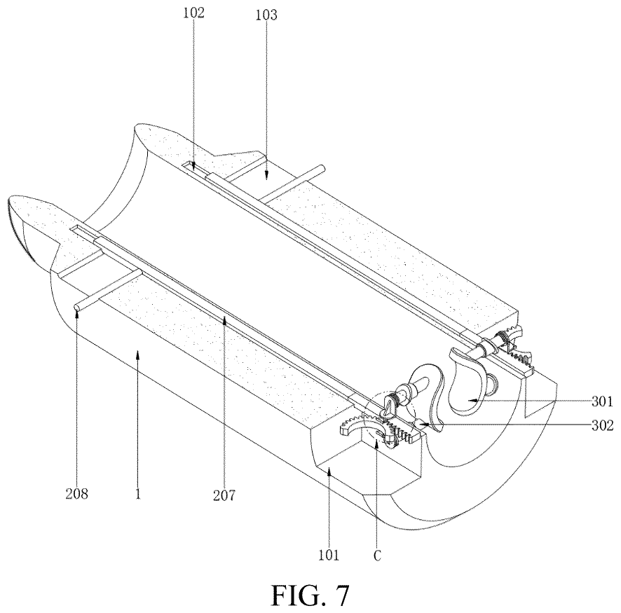

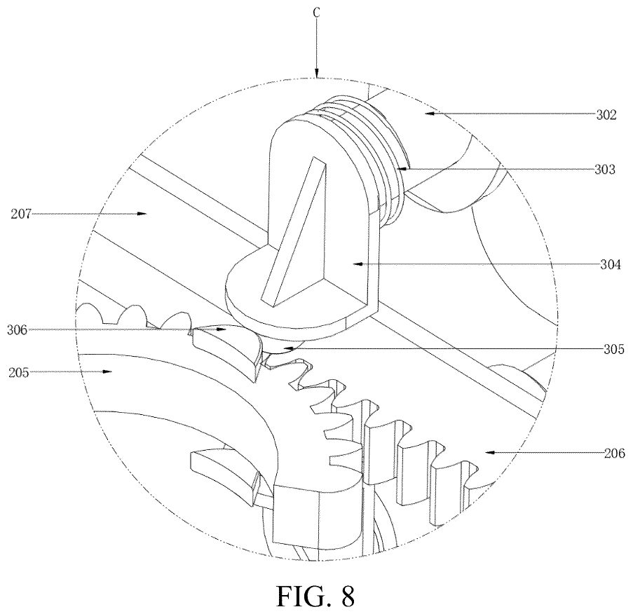

As shown in FIG. 1 to FIG. 8, the present disclosure provides an embodiment: An intubation tube holder includes:

a clamping sleeve 1. A mounting notch 101 is provided in each of two sides of one end of the clamping sleeve 1. The two mounting notches 101 are symmetrically distributed about a circle center of the clamping sleeve 1. A support plate 202 is rotatably connected into each of the two mounting notches 101 elastic dental pads 203 are fixedly connected to upper and lower ends of the support plates 202.

Hinge bars 201 are vertically rotatably connected into the mounting notches 101. One end of each support plate 202 is fixedly connected to a side wall of each hinge bar 201. Torsional springs 204 sleeve the hinge bars 201 at the upper and lower ends of the support plates 202. One end of each of the two torsional springs 204 close to each support plate 202 is fixedly connected to the support plate 202, and the other end is respectively fixedly connected to a top wall and bottom wall of each mounting notch 101.

Curved gears 205 are fixedly connected to side walls of the two hinge bars 201. Racks 206 are meshed with peripheries of the two curved gears 205. The two racks 206 are both located between the curved gears 205 and inner walls of the mounting notches 101. First sliding chutes 102 communicated with the mounting notches 101 are provided inside side walls of two sides of the clamping sleeve 1 in a length direction of the clamping sleeve 1. Coupling bars 207 are slidably connected in the two first sliding chutes 102. End portions of the two linkage bars 207 are fixedly connected to end portions of the racks 206 corresponding to the two linkage bars 207.

To wear this intubation tube holder, first of all, a medical staff opens the mouth of a patient and pulls the two linkage bars 207 to slide along the first sliding chutes 102, which further drives the two racks 206 to move. When moving, the two racks 206 drive the curved gears 205 meshed with the two racks 206 to rotate, so as to drive the two support plates 202 to rotate along a hinge position between the hinge bars 201. In this case, the torsional springs 204 undergo torsional deformation for force storage, so that the two symmetrically distributed support plates 202 and elastic dental pads 203 are contracted to the middle, and it is convenient to place the holder in the oral cavity of the patient, thereby improving the convenience of mounting of the holder. After this holder is placed into the oral cavity of the patient, an intubation tube is immediately threaded through the clamping sleeve 1 and inserted into the oral cavity of the patient. Later, a pulling force on the linkage bars 207 is withdrawn, so that the torsional springs 204 are elastically released to drive the support plates 202 to rotate towards the outside to original positions along connection positions for the hinge bars 201 and the mounting notches 101. The two support plates 202 move to a periphery to drive the elastic dental pads 203 to move, so that the elastic dental pads 203 on the two sides are just located between upper and lower groups of chewing teeth of the patient. When the medical staff withdraws the opening force on the mouth of the patient, the mandible muscles of the patient naturally contract, causing the upper and lower chewing teeth of the patient to be just in contact with the elastic dental pads 203 at tops and bottoms of the support plates 202. The elastic dental pads 203 play a role of supporting the mouth of the patient. Compared with an existing holder, the intubation tube holder solves the following problem that the patient bites on an outer wall of the holder with the front teeth for a long time, and further avoids looseness and deformation due to overtime stress on the front teeth of the patient.

In an embodiment of the present disclosure, one end, away from each rack 206, of each of the two linkage bars 207 is fixedly connected with a lever 208, and the levers 208 are perpendicular to the linkage bars 207. Second sliding chutes 103 communicated to insides of the first sliding chutes 102 are provided in two sides of a side wall of the clamping sleeve 1. The two levers 208 are slidably connected into the corresponding second sliding chutes 103. Outer side ends of the two levers 208 extend to outer sides of the second sliding chutes 103. The outer side ends of the two levers 208 are jointly connected with one arc-shaped connection frame 209. The arc-shaped connection frame 209 is located at a top periphery of the clamping sleeve 1.

The medical staff pulls the arc-shaped connection frame 209 to drive the two levers 208 to slide along the second sliding chutes 103, and the two levers 208 slide to drive the two linkage bars 207 to slide along the first sliding chutes 102.

In an embodiment of the present disclosure, the two support plates 202 are symmetrically distributed about the circle center of the clamping sleeve 1. The two support plates 202 are arranged in an arc manner. It is convenient to abut against the teeth.

In an embodiment of the present disclosure, the elastic dental pads 203 are made of a soft silicon material.

In an embodiment of the present disclosure, two symmetrically distributed clamping blocks 301 are provided inside the clamping sleeve 1. Two clamping ejector rods 302 distributed above and below are fixedly connected to side surfaces of the two clamping blocks 301 far away from each other. The two clamping ejector rods 302 slide through the clamping sleeve 1, and extend into the mounting notches 101. One end, away from each clamping block 301, of each of the two clamping ejector rods 302 is fixedly connected with a thrust base 304. One side surface of each thrust base 304 close to the clamping block 301 is fixedly connected with a return spring 303. Each return spring 303 sleeves a periphery of each clamping ejector rod 302. One end of each return spring 303 away from each thrust base 304 is fixedly connected to a side wall of each mounting notch 101.

Upper and lower side surfaces, close to each other, of the two thrust bases 304 on the same side are rotatably connected with guide wheels 305. Upper and lower ends of the curved gears 205 are fixedly connected with cams 306. The two cams 306 are respectively in one-to-one correspondence to positions of the two guide wheels 305.

When the support plates 202 are driven to rotate towards two sides through the torsional springs 204, the cams 306 connected to the curved gears 205 are driven to rotate together, so that the cams 306 push the guide wheels 305 to move towards the middle of the clamping sleeve 1. The movement of the guide wheels 305 drives the thrust bases 304 and the clamping ejector rods 302 to move towards the middle along slidable connection positions of the clamping sleeve 1, which further pushes the clamping blocks 301 to move, so that the two symmetrically distributed clamping blocks 301 move towards the middle and then automatically complete clamping on the intubation tube. Compared with the existing holder, the present disclosure greatly improves ease of use of this holder.

In an embodiment of the present disclosure, the two clamping blocks 301 are symmetrically distributed about the circle center of the clamping sleeve 1, and the two clamping blocks 301 are arranged in an arc manner. It is convenient to abut against and clamp an outer wall of a catheter. In an embodiment of the present disclosure, a water supplement pressure plate 401 is slidably connected to the periphery of the clamping sleeve 1. One side surface of the water supplement pressure plate 401 close to the support plates 202 is fixedly connected with a rubber bag 402 for water supplement. The rubber bag 402 for water supplement is located at an edge of the water supplement pressure plate 401. Several small holes 403 for water supplement are provided in one side surface of the rubber bag 402 for water supplement close to the support plates 202. The several small holes 403 for water supplement are communicated to an inside of the rubber bag 402 for water supplement. The rubber bag 402 for water supplement is filled with a water absorption sponge block 404.

A circular slot 405 is provided in an upper part of the water supplement pressure plate 401 in a penetrating manner. A water injection tube 406 is fixedly connected in the circular slot 405. A water supplement hole coaxial with the circular slot 405 is provided in an upper part of one side surface of the rubber bag 402 for water supplement close to the water supplement pressure plate 401. An inner diameter of the water supplement hole is less than an inner diameter of the circular slot 405. An inner side end of the water injection tube 406 is fixedly connected to the rubber bag 402 for water supplement around the water supplement hole. A rubber plug 407 is embedded into the water injection tube 406.

When the lips of the patient are dry during treatment, a syringe can be used to extract pure water, and a needle tube of the syringe is threaded through the rubber plug 407 to inject the pure water into the rubber bag 402 for water supplement. The pure water injected into the rubber bag 402 for water supplement is immediately absorbed by the water absorption sponge block 404 and infiltrates the entire water absorption sponge block 404, so that the water absorption sponge block 404 becomes a wet state. Later, the syringe is removed, and the water supplement pressure plate 401 is pushed to move, to drive the rubber bag 402 for water supplement and the water absorption sponge block 404 to move together, until the rubber bag 402 for water supplement is in contact with the lips of the patient. The rubber bag 402 for water supplement is compressed to elastically collapse to press the water absorption sponge block 404 inside the rubber bag 402 for water supplement, and the pure water contained inside the water absorption sponge block 404 is extruded out through the plurality of small holes 403 for water supplement, to moisturize the lips of the patient. Compared with water supplement by using a cotton swab, this greatly improves convenience and efficiency.

In an embodiment of the present disclosure, the water supplement pressure plate 401 is arranged in an arc manner. It is convenient to abut against the lips.

In an embodiment of the present disclosure, a plurality of rebound seats 501 distributed at equal angles are fixedly connected to an outer wall of the clamping sleeve 1. The rebound seats 501 are located on one side of the water supplement pressure plate 401 away from the rubber bag 402 for water supplement. One side surface of each of the plurality of rebound seats 501 close to the water supplement pressure plate 401 is fixedly connected with a rebound rubber band 502. One end, away from each rebound seat 501, of each of the plurality of rebound rubber bands 502 is fixedly connected to a surface of the water supplement pressure plate 401.

The water supplement pressure plate 401 is pushed to slide towards the lips of the patient. At the same time, the plurality of rebound rubber bands 502 are elastically pulled. After the moisturizing on the lips of the patient is completed, the water supplement pressure plate 401 is loosened, and a rebound force of the plurality of rebound rubber bands 502 drives the water supplement pressure plate 401 and the rubber bag 402 for water supplement to slide and return along the outer wall of the clamping sleeve 1, thereby automatically releasing pressure of the rubber bag 402 for water supplement on the lips of the patient. Manual resetting is not required, so that ease of use of this holder is further improved.

In an embodiment of the present disclosure, the rubber bag (402) for water supplement is made of a highly elastic rubber material.

Working principle: To wear this intubation tube holder, first of all, a medical staff opens the mouth of a patient and pulls the arc-shaped connection frame 209 to drive the two levers 208 to slide along the second sliding chutes 103. The two levers 208 slide to drive the two linkage bars 207 to slide along the first sliding chutes 102, which further drives the two racks 206 to move. When moving, the two racks 206 drive the curved gears 205 meshed with the two racks 206 to rotate, so as to drive the two support plates 202 to rotate along a hinge position between the hinge bars 201, so that the two symmetrically distributed support plates 202 and elastic dental pads 203 are contracted towards the middle, which facilitates placement of this holder into the oral cavity of the patient. During this period, the torsional springs 204 undergo torsional deformation for force storage. When this holder is placed into the oral cavity of the patient, an intubation tube is immediately threaded through the clamping sleeve 1 and inserted into the oral cavity of the patient, and a bandage is used to fix the clamping sleeve 1. Later, a pulling force on the arc-shaped connection frame 209 is withdrawn, so that the torsional springs 204 are elastically released to drive the support plates 202 to rotate towards the outside to original positions along connection positions for the hinge bars 201 and the mounting notches 101. The two support plates 202 move to a periphery to drive the elastic dental pads 203 to move, so that the elastic dental pads 203 on the two sides are just located between upper and lower groups of chewing teeth of the patient. When medical staff withdraws the opening force on the mouth of the patient, the mandible muscles of the patient naturally contract, causing the upper and lower chewing teeth of the patient to be just in contact with the elastic dental pads 203 at tops and bottoms of the support plates 202. The elastic dental pads 203 play a role of supporting the mouth of the patient. Compared with an existing holder, the intubation tube holder solves the following problem that the patient bites on an outer wall of the holder with the front teeth for a long time, and further avoids looseness and deformation due to overtime stress on the front teeth of the patient.

When the support plates 202 are driven to rotate towards two sides through the torsional springs 204, the cams 306 connected to the curved gears 205 are driven to rotate together, so that the cams 306 push the guide wheels 305 to move towards the middle of the clamping sleeve 1. The movement of the guide wheels 305 drives the thrust bases 304 and the clamping ejector rods 302 to move towards the middle along slidable connection positions of the clamping sleeve 1, which further pushes the clamping blocks 301 to move, so that the two symmetrically distributed clamping blocks 301 move towards the middle and then automatically complete clamping on the intubation tube. Compared with the existing holder, the present disclosure greatly improves ease of use of this holder.

When the lips of the patient are dry during treatment, a syringe can be used to extract pure water, and a needle tube of the syringe is threaded through the rubber plug 407 to inject the pure water into the rubber bag 402 for water supplement. The pure water injected into the rubber bag 402 for water supplement is immediately absorbed by the water absorption sponge block 404 and infiltrates the entire water absorption sponge block 404, so that the water absorption sponge block 404 becomes a wet state. Later, the syringe is removed, and the water supplement pressure plate 401 is pushed towards a direction of supporting a component. At the same time, the plurality of rebound rubber bands 502 are elastically pulled, and the water supplement pressure plate 401 moves to drive the rubber bag 402 for water supplement and the water absorption sponge block 404 to move together, until the rubber bag 402 for water supplement is in contact with the lips of the patient. The rubber bag 402 for water supplement is compressed to elastically collapse to press the water absorption sponge block 404 inside the rubber bag 402 for water supplement, and the pure water contained inside the water absorption sponge block 404 is extruded out through the plurality of small holes 403 for water supplement, to moisturize the lips of the patient. Compared with water supplement by using a cotton swab, this greatly improves convenience and efficiency. After the moisturizing on the lips of the patient is completed, the water supplement pressure plate 401 is loosened, and a rebound force of the plurality of rebound rubber bands 502 drives the water supplement pressure plate 401 and the rubber bag 402 for water supplement to slide and return along the outer wall of the clamping sleeve 1, thereby automatically releasing pressure of the rubber bag 402 for water supplement on the lips of the patient.

In the descriptions of this specification, a description of a reference term such as “an embodiment”, “an example”, or “a specific example” means that a specific feature, structure, material, or characteristic that is described with reference to the embodiment or the example is included in at least one embodiment or example of the present disclosure. In this specification, exemplary descriptions of the foregoing terms do not necessarily refer to the same embodiment or example. In addition, the described specific features, structures, materials, or characteristics may be combined in a proper manner in any one or more of the embodiments or examples.

The foregoing displays and describes basic principles, main features of the present disclosure and advantages of the present disclosure. Technicians in this industry should understand that the present disclosure is not limited by the embodiments described above. The embodiments described above and the specification only describe the principles of the present disclosure. The present disclosure will also have various changes and improvements without departing from the spirit and scope of the present disclosure, and these changes and improvements shall all fall within the claimed scope of the present disclosure.

What is claimed is:

1. An intubation tube holder, comprising:
a clamping sleeve (1), wherein a mounting notch (101) is provided in each of two sides of one end of the clamping sleeve (1); the two mounting notches (101) are symmetrically distributed about a circle center of the clamping sleeve (1); a support plate (202) is rotatably connected into each of the two mounting notches (101) elastic dental pads (203) are fixedly connected to upper and lower ends of the support plates (202);
hinge bars (201) are vertically rotatably connected into the mounting notches (101); one end of each support plate (202) is fixedly connected to a side wall of each hinge bar (201); torsional springs (204) sleeve the hinge bars (201) at the upper and lower ends of the support plates (202); one end of each of the two torsional springs (204) close to each support plate (202) is fixedly connected to the support plate (202), and the other end is respectively fixedly connected to a top wall and bottom wall of each mounting notch (101);
curved gears (205) are fixedly connected to side walls of the two hinge bars (201); racks (206) are meshed with peripheries of the two curved gears (205); the two racks (206) are both located between the curved gears (205) and inner walls of the mounting notches (101); first sliding chutes (102) communicated with the mounting notches (101) are provided inside side walls of two sides of the clamping sleeve (1) in a length direction of the clamping sleeve (1); linkage bars (207) are slidably connected in the two first sliding chutes (102); end portions of the two linkage bars (207) are fixedly connected to end portions of the racks (206) corresponding to the two linkage bars (207);
the two support plates (202) are symmetrically distributed about the circle center of the clamping sleeve (1); the two support plates (202) are arranged in an arc manner;
the elastic dental pads (203) are made of a soft silicon material.

2. The intubation tube holder according to claim 1, wherein two symmetrically distributed clamping blocks (301) are provided inside the clamping sleeve (1); two clamping ejector rods (302) distributed above and below are fixedly connected to side surfaces of the two clamping blocks (301) far away from each other; the two clamping ejector rods (302) slide through the clamping sleeve (1), and extend into the mounting notches (101); one end, away from each clamping block (301), of each of the two clamping ejector rods (302) is fixedly connected with a thrust base (304); one side surface of each thrust base (304) close to the clamping block (301) is fixedly connected with a return spring (303); each return spring (303) sleeves a periphery of each clamping ejector rod (302); one end of each return spring (303) away from each thrust base (304) is fixedly connected to a side wall of each mounting notch (101);
upper and lower side surfaces, close to each other, of the two thrust bases (304) on the same side are rotatably connected with guide wheels (305); upper and lower ends of the curved gears (205) are fixedly connected with cams (306); and the two cams (306) are respectively in one-to-one correspondence to positions of the two guide wheels (305).

3. The intubation tube holder according to claim 2, wherein the two clamping blocks (301) are symmetrically distributed about the circle center of the clamping sleeve (1), and the two clamping blocks (301) are arranged in an arc manner.

4. The intubation tube holder according to claim 1, wherein a water supplement pressure plate (401) is slidably connected to the periphery of the clamping sleeve (1); one side surface of the water supplement pressure plate (401) close to the support plates (202) is fixedly connected with a rubber bag (402) for water supplement; the rubber bag (402) for water supplement is located at an edge of the water supplement pressure plate (401); several small holes (403) for water supplement are provided in one side surface of the rubber bag (402) for water supplement close to the support plates (202); the several small holes (403) for water supplement are communicated to an inside of the rubber bag (402) for water supplement; the rubber bag (402) for water supplement is filled with a water absorption sponge block (404);
a circular slot (405) is provided in an upper part of the water supplement pressure plate (401) in a penetrating manner; a water injection tube (406) is fixedly connected in the circular slot (405); a water supplement hole coaxial with the circular slot (405) is provided in an upper part of one side surface of the rubber bag (402) for water supplement close to the water supplement pressure plate (401); an inner diameter of the water supplement hole is less than an inner diameter of the circular slot (405); an inner side end of the water injection tube (406) is fixedly connected to the rubber bag (402) for water supplement around the water supplement hole; and a rubber plug (407) is embedded into the water injection tube (406).

5. The intubation tube holder according to claim 4, wherein the water supplement pressure plate (401) is arranged in an arc manner.

6. The intubation tube holder according to claim 4, wherein a plurality of rebound seats (501) distributed at equal angles are fixedly connected to an outer wall of the clamping sleeve (1); the rebound seats (501) are located on one side of the water supplement pressure plate (401) away from the rubber bag (402) for water supplement; one side surface of each of the plurality of rebound seats (501) close to the water supplement pressure plate (401) is fixedly connected with a rebound rubber band (502); one end, away from each rebound seat (501), of each of the plurality of rebound rubber bands (502) is fixedly connected to a surface of the water supplement pressure plate (401).

7. The intubation tube holder according to claim 4, wherein the rubber bag (402) for water supplement is made of a highly elastic rubber material.
